(19) United States
(12) Patent Application Publication
(10) Pub. No.: US 2024/0127944 A1
(43) Pub. Date: Apr. 18, 2024

(54) FATIGUE DATA GENERATION SYSTEM AND FATIGUE DATA GENERATION METHOD

(71) Applicant: INSTITUTE FOR INFORMATION INDUSTRY, TAIPEI (TW)

(72) Inventors: Wen-Chien HUANG, TAIPEI (TW); Hong-En CHEN, TAIPEI (TW); Hsiao-Chen CHANG, TAIPEI (TW); Jing-Ming CHIU, TAIPEI (TW)

(21) Appl. No.: 18/056,701

(22) Filed: Nov. 17, 2022

(30) Foreign Application Priority Data
Oct. 14, 2022 (TW) 111139111

(51) Int. Cl. G16H 40/63

(52) U.S. Cl. CPC G16H 40/63

(57) ABSTRACT

A fatigue data generation method, comprising: obtaining, by the camera device, a target image; obtaining, by a processor, a target feature data from the target image, and inputting the target feature data to a fatigue analysis model which stored in a storage unit, wherein the fatigue analysis model comprises a plurality of reference physiological signals, a plurality of reference feature data, a plurality of reference fatigue data and a plurality of correlation parameters; and generating a target fatigue data according to the target feature data, the plurality of reference feature data and the plurality of correlation parameters.

### CROSS-REFERENCE TO RELATED APPLICATION

This application claims priority to Taiwan Application Serial Number 111139111, filed Oct. 14, 2022, which is herein incorporated by reference in its entirety.

### BACKGROUND

### Technical Field

The present disclosure relates to a data generation technology, especially a fatigue data generation system and fatigue data generation method.

### Description of Related Art

For many industries, the physiological state of workers directly affects the quality of their work, and even determines product quality or production efficiency. Therefore, it is necessary to detect the physiological state of the worker. However, most of the detection is to obtain physiological signals by means of contact, and the physiological signals must be analyzed in order to more accurately estimate the corresponding physiological state. Therefore, the detection cannot obtain the analysis results quickly, resulting in some limitations in application.

### SUMMARY

One aspect of the present disclosure is a fatigue data generation method, comprising: obtaining, by a camera device, a target image; obtaining, by a processor, a target feature data from the target image, and inputting the target feature data to a fatigue analysis model which stored in a storage unit, wherein the fatigue analysis model comprises a plurality of reference physiological signals, a plurality of reference feature data, a plurality of reference fatigue data and a plurality of correlation parameters; and generating a target fatigue data according to the target feature data, the plurality of reference feature data and the plurality of correlation parameters.

Another aspect of the present disclosure is a fatigue data generation system, comprising a camera device, a storage unit and a processor. The camera device is configured to obtain a target image. The storage unit is configured to store a fatigue analysis model. The fatigue analysis model comprises a plurality of reference physiological signals, a plurality of reference feature data, a plurality of reference fatigue data and a plurality of correlation parameters. The processor is communicatively connected to the camera device and the storage unit, and configured to: receive the target image to obtain a target feature data from the target image; and generate a target fatigue data according to the target feature data, the plurality of reference feature data, the plurality of reference physiological signals and the plurality of correlation parameters.

It is to be understood that both the foregoing general description and the following detailed description are by examples, and are intended to provide further explanation of the disclosure as claimed.

### BRIEF DESCRIPTION OF THE DRAWINGS

The present disclosure can be more fully understood by reading the following detailed description of the embodiment, with reference made to the accompanying drawings as follows:

FIG. 1 is a schematic diagram of a fatigue data generation system in some embodiments of the present disclosure.

FIG. 2 is a schematic diagram of a reference image in some embodiments of the present disclosure.

FIG. 3 is a flowchart illustrating the method of establishing fatigue analysis model in some embodiments of the present disclosure.

FIG. 4 is a flowchart illustrating the fatigue data generation method in some embodiments of the present disclosure.

FIG. 5A-5B are schematic diagrams of the target image and the target feature data in some embodiments of the present disclosure.

### DETAILED DESCRIPTION

For the embodiment below is described in detail with the accompanying drawings, embodiments are not provided to limit the scope of the present disclosure. Moreover, the operation of the described structure is not for limiting the order of implementation. Any device with equivalent functions that is produced from a structure formed by a recombination of elements is all covered by the scope of the present disclosure. Drawings are for the purpose of illustration only, and not plotted in accordance with the original size.

It will be understood that when an element is referred to as being “connected to” or “coupled to”, it can be directly connected or coupled to the other element or intervening elements may be present. In contrast, when an element to another element is referred to as being “directly connected” or “directly coupled”, there are no intervening elements present. As used herein, the term “and/or” includes an associated listed items or any and all combinations of more.

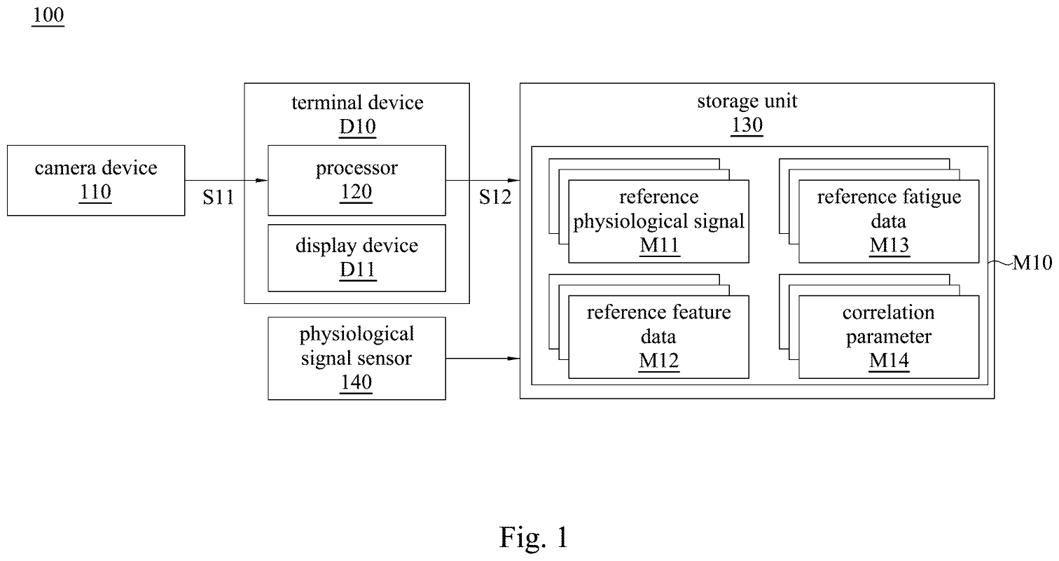

FIG. 1 is a schematic diagram of a fatigue data generation system 100 in some embodiments of the present disclosure. The fatigue data generation system 100 is configured to obtain/capture image of a target human body (e.g., workers, drivers), so as to analyze the current fatigue level of the target human body.

The fatigue data generation system 100 includes a camera device 110, a processor 120 and a storage unit 130. The camera device 110 can be implemented to a camera or a video device with camera, and is configured to obtain/capture image of the target human body, so as to generate a target image S11 (can be photo or video).

The processor 120 is communicatively connected to the camera device 110 to receive the target image S11, and obtains a target feature data S12 from the target image S11, such as by image recognition. In some embodiments, the processor 120 is arranged in a terminal device D10. The terminal device D10 can be implemented to a detection host device near the target human body (e.g., a microprocessor in a communication device), or a remote server (e.g., a cloud server connected to the communication device).

The storage unit 130 is communicatively connected to the processor 120, and stores a fatigue analysis model M10. The fatigue analysis model M10 includes multiple reference physiological signals M11, multiple reference feature data M12, multiple reference fatigue data M13 and multiple correlation parameters M14. The reference physiological signals M11 can be an electromyography (EMG) signal, but the present disclosure is not limited to this. The reference physiological signals may includes at least one of an electromyogram, an electrocardiogram, a heart rate, a muscle strength and a blood pressure. The reference feature data M12 includes image features of the target human body, it will be detailed in the following paragraphs. The reference fatigue data M13 may be a value or a ratio defined by the storage unit 130, and its amount corresponds to the fatigue level of the target human body (e.g., when it is greater than a first threshold, the fatigue level is “60%”).

The correlation parameters M14 is configured to establish multiple first correspondences between reference physiological signals M11 and the reference feature data M12, and establish multiple second correspondences between reference physiological signals M11 and the reference fatigue data M13. Specifically, the processor 120 is communicatively connected to the storage unit 130, and integrated the reference physiological signals M11, the reference feature data M12 and the reference fatigue data M13 into a fatigue analysis model M10 according to the correlation parameters M14.

It should be mentioned here that although in FIG. 1, the processor 120 is arranged in the terminal device D10, and the terminal device D10 and the storage unit 130 are shown as two independent devices, the present disclosure does not limited to this. In other embodiments, the processor 120 and the storage unit 130 can also be integrated into the same device. For example, the processor 120 and the storage unit 130 are both arranged in a server, and the processor 120 can be a central processing unit (CPU), a microprocessor (MCU), or other devices with data access, data calculation or circuits or components of similar function. The storage unit 130 can be implemented as non-volatile memory, volatile memory, random access memory, write-only memory, flash memory, electronically erasable rewritable read-only memory, and other types of memory, or a combination of the above.

After the processor 120 generates the target feature data S12, the processor 120 inputs the target feature data S12 into the fatigue analysis model M10, so as to analyze the target feature data S12 by the reference feature data M12 and the correlation parameters M14 of the fatigue analysis model M10 and calculate a “target fatigue data”. The target fatigue data is configured to estimate a current fatigue state of the target human body. In some other embodiments, the processor 120 generates the target fatigue data according to the target feature data S12, the reference feature data M12, the reference physiological signals M11 and the correlation parameters M14. In one embodiment, the fatigue data generation system 100 can display the target fatigue data in the form of numbers or colors (lights) by a display device D11 of the terminal device D10.

The present disclosure combines “contact” and “contactless” detection methods to build and train the fatigue analysis model M10. Accordingly, when the fatigue data generation system 100 is actually used to detect the fatigue level of the target human body, the fatigue data generation system 100 can only obtain/capture the target image in a “contactless” detection method, so as to estimate the current fatigue level of the target human body by the fatigue analysis model M10.

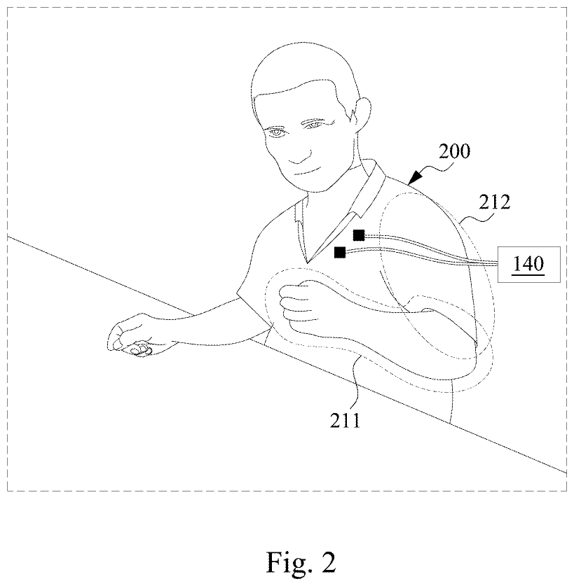

FIG. 2 is a schematic diagram of the application of the fatigue data generation system 100 in some embodiments of the present disclosure. The following describes how to create the “the fatigue analysis model”. As shown in FIG. 1 and FIG. 2, the fatigue data generation system 100 further includes a physiological signal sensor 140. The physiological signal sensor 140 is arranged on the target human body 200 in contact (e.g., contact electrodes), and is communicatively connected to the storage unit 130 and the processor 120, so as to detect/receive contact signals and establish the reference physiological signals. For example, the physiological signal sensor 140 transmits the contact signals to the processor 120, and establish the reference physiological signals in the storage unit 130 by the processor 120. In one embodiment, the physiological signal sensor 140 includes at least one of an electromyogram sensor, an electrocardiogram sensor, a heart rate sensor, a muscle strength sensor and a blood pressure sensor. Signals detected by the physiological signal sensor 140 belong “the reference physiological signals”.

As mentioned above, when building the fatigue analysis model M10, the camera device 110 is configured to capture a reference image (contactless) of the target human body 200. The reference image will be analyzed (e.g., image recognition by the processor 120) to be establish as “reference feature data”.

The processor 120 determines a detection time of each reference physiological signal M11 and each reference feature data M12, so as to establish first correspondences between the reference feature data M12 and the reference physiological signals M11 in the storage unit 130 according to the reference feature data M12 and the reference physiological signals M11 corresponding to a same time. For example, if at the same time, the processor 120 receives a reference physiological signal M11 (e.g., heart rate is 75 bpm) and a reference feature data M12 (e.g., an image of the target), the processor 120 establishes/creates a correspondence between “75 bpm” and “the image of the target”.

In addition, the processor 120 further uses a conversion formula to create second correspondences between the reference physiological signals M11 and the reference fatigue data M13 in the storage unit 130. For example, one of the reference physiological signals M11 is “heart rate 75 bpm”, and the normal heart rate range is “60-100 bpm”, then the processor 120 may define the reference fatigue data M13 corresponding to the reference physiological signals M11 of “heart rate 75 bpm” as “37.5%”. In one embodiment, the conversion formula is established based on all reference physiological signals M11. For example, determine the physiological state range of the target human body 200 (e.g., the corresponding range from normal state to fatigue state is 60 bpm-120 bpm), and then organize it into a numerical or proportional conversion calculation formula. The present disclosure is not limited to this, the conversion formula can be adjusted according to actual needs.

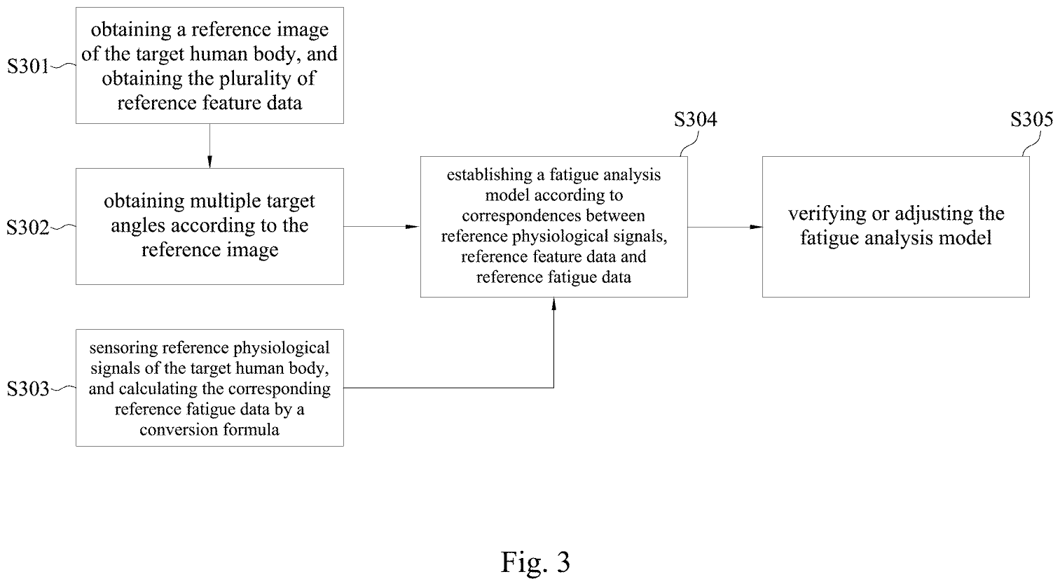

FIG. 3 is a flowchart illustrating the method of establishing fatigue analysis model M10 in some embodiments of the present disclosure. In step S301, the camera device 110 obtains/captures a reference image of the target human body 200, and the processor 120 obtains the reference feature data M12 from the reference image. In some embodiments, the reference feature data M12 includes multiple reference nodes of the reference image. The processor 120 is configured to identify the reference image to obtain the reference feature data M12 (or reference nodes). The reference nodes is configured to define at least one reference part of the target human body 200 (e.g., forearm, upper arm or neck).

In step S302, the processor 120 is further configured to obtain multiple reference angles according to the reference image. The reference angle is calculated from a relative angle between multiple adjacent reference parts of the target human body 200. For example, the processor 120 identifies at least four reference nodes NA, NB, NC, and ND from the reference image, wherein a connection line between the reference nodes NA and NB is defined as one reference part “forearm”. A connection line between the reference nodes NC and ND is defined as one reference part “upper arm”. A angle between the two connection lines (i.e., two reference parts “the forearm, the upper arm”) is the reference angle.

In step S303, the physiological signal sensor 140 detects the reference physiological signals M11 of the target human body 200. After the processor 120 receives the reference physiological signals M11, the processor 120 calculates the corresponding reference fatigue data M13 by a conversion formula, so as to establish correspondences between the reference physiological signals M11 and the reference fatigue data M13 in the storage unit 130.

After the processor 120 receives the reference physiological signals M11, the reference feature data M12 and the reference fatigue data M13 though steps S301-S303, in step S304, the processor 120 establishes first correspondences between the reference feature data M12 and the reference physiological signals M11, and establishes second correspondences between the reference physiological signals M11 and the reference fatigue data M13, so as to establish/generate the fatigue analysis model M10 in the storage unit 130.

In step S305, the processor 120 repeats the above steps S301-S304, accumulates a large amount of training data, and verify or adjust the fatigue analysis model M10. In some embodiments, the fatigue data generation system 100 verifies the reference fatigue data M13 to determine whether to adjust the definition of the reference fatigue data M13, or to determine whether to adjust the second correspondences between the reference physiological signals M11 and the reference fatigue data M13.

When the fatigue data generation system 100 establish the fatigue analysis model M10, the fatigue data generation system 100 uses both of “contact data” and “contactless data (image)”. Therefore, the fatigue analysis model M10 ensures analysis accuracy, and in the subsequent actual analysis, the fatigue data generation system 100 can only use “contactless data (image)” to estimate the fatigue level, and does not need to detect the contact data again, to ensure the speed and efficiency of the analysis.

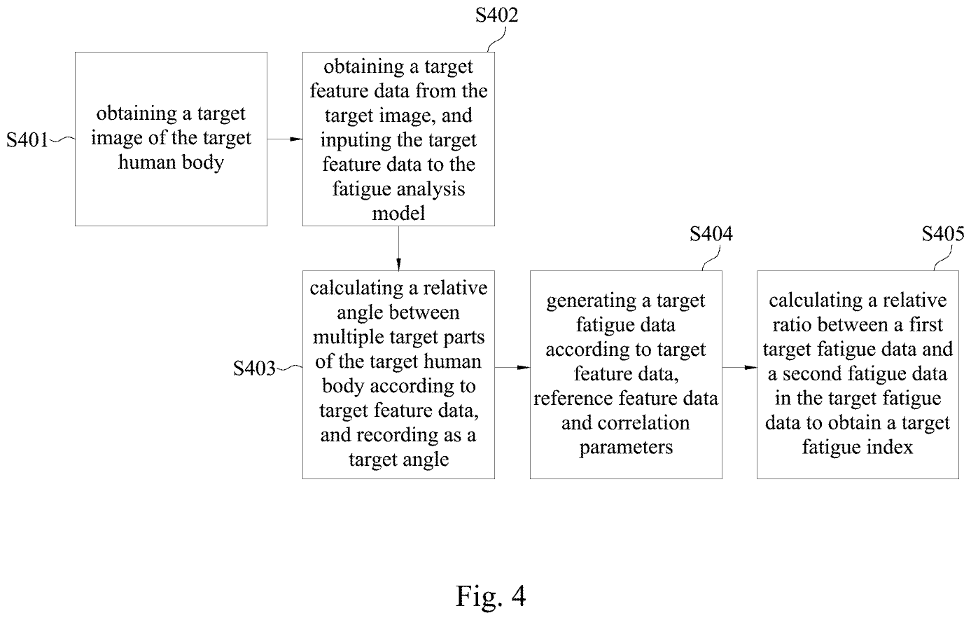

The following describes how the fatigue data generation system 100 actually performs detection/estimation. FIG. 4 is a flowchart illustrating the fatigue data generation method in some embodiments of the present disclosure. In step S401, the camera device 110 obtains the target image S11 of the target human body 200, and transmits the target image S11 to the processor 120 by wire or wirelessly.

In step S402, the processor 120 obtains the target feature data S12 from the target image S11, amd inputs the target feature data S12 into the fatigue analysis model M10 in the storage unit 130. As above embodiment, the fatigue analysis model M10 includes multiple reference physiological signals M11, multiple reference feature data M12, multiple reference fatigue data M13 and multiple correlation parameters M14. The correlation parameters M14 is configured to multiple correspondences between the reference physiological signals M11, the reference feature data M12 and the reference fatigue data M13 corresponding to the same time.

In step S403, the processor 120 further calculates a relative angle between multiple target parts 210 of the target human body 200 according to target feature data S12, and records the relative angle as a target angle. In other words, the processor 120 calculate at least one target angle according to multiple target parts of a target human body 200 in the target image S11. In some embodiments, “the target angle” can be a angle between the centerlines of two adjacent target parts.

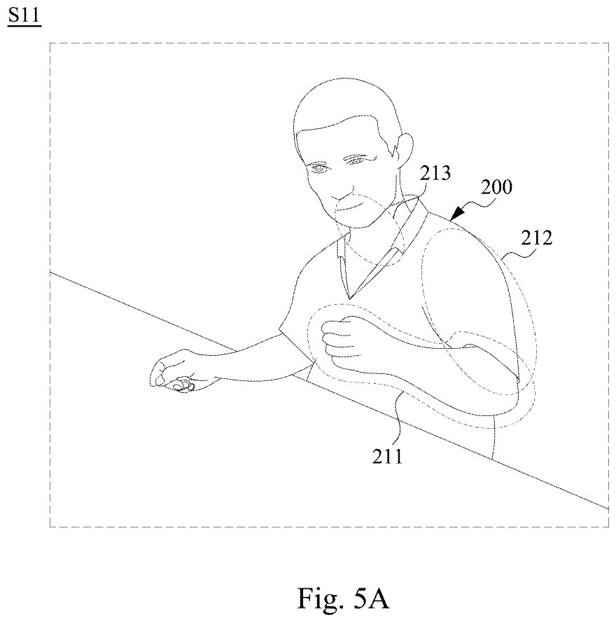

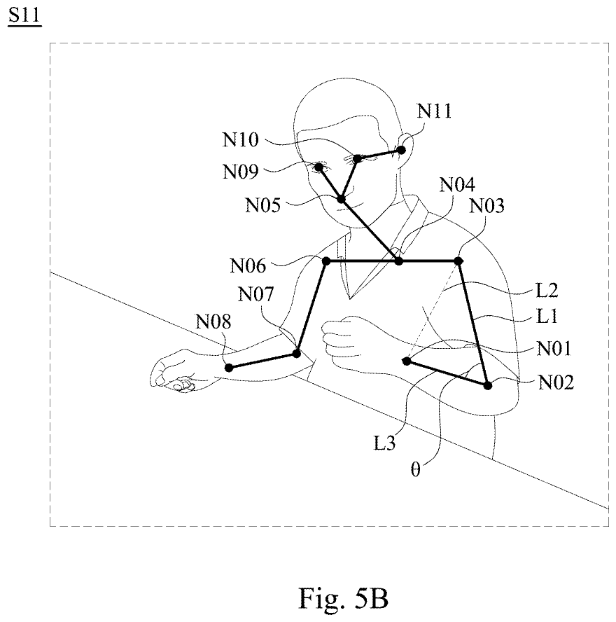

FIG. 5A-5B are schematic diagrams of the target image S11 and the target feature data S12 in some embodiments of the present disclosure. In one embodiment, the processor 120 uses image recognition to identify multiple target parts of the target human body 200 in the target image S11, and then calculates the target angle. In some embodiments, the target parts can be the same as the reference parts used in establishing the fatigue analysis model M10. In other words, the processor 120 can identify the target image S11 according to the reference parts.

Specifically, the processor 120 identifies the target image S11 first by image recognition to generate a human body skeleton. The human body skeleton includes multiple part nodes N01-N11 and multiple corresponding part coordinates in the target image S11. The part nodes N01-N11 and the part coordinates are configured to define multiple target parts. The processor 120 generates at least one target angle according to at least a part of the part nodes N01-N11 and the corresponding part coordinates, and uses the target angle as the target feature data S12. As shown in the figure, the part nodes N01 and N02 are configured to define a target part 211 “foreground”. The part nodes N02, N03 are configured to define a target part 212 “upper arm”. The part nodes N04 and N05 are configured to define a target part 213 “neck”. The angle between the target part 211 and the target part 212 is the target angle (e.g., 60 degrees).

In some other embodiments, the processor 120 calculates the part nodes and the corresponding part coordinates by Law of Cosines, so as to generate the target angle. As shown in FIG. 5B, the connection line L1 of the part nodes N02 and the part nodes N03, the connection line L2 of the part nodes N03 and the part nodes N01, the connection line L3 of the part nodes N01 and the part nodes N02 are configured to form a triangle. The angle θ between the lines L1 and L3 can be calculated by using the law of cosines, as shown in the following formula:

L2²=L1²+L3²−2×L1×L3×cos θ

In step S404, the processor 120 uses the fatigue analysis model M10 to generate the target fatigue according to the target feature data S12, the reference feature data M12 and the correlation parameters M14 (i.e., estimate the current the fatigue level of the target human body 200). Specifically, the processor 120 compares the target feature data S12 with all the reference feature data M12 to select at least one similar feature data among all the reference feature data M12 (e.g., at least one reference feature data M12 where the angle between the forearm and the upper arm is close to 60 degrees).

As mentioned above, according to the selected similar feature data, the processor 120 further selects the corresponding correlation parameters M14 and a part of the reference physiological signals M11 to generate a control physiological data. For example, the target feature data S12 is “target angle between the forearm and the upper arm is 60 degrees”. The two similar feature data selected by the processor 120 are respectively “target angle between the forearm and the upper arm is 55 degrees” and “target angle between the forearm and the upper arm is 65 degrees”. The reference physiological signals corresponding to these two similar feature data are “heart rate 70 bpm” and “heart rate 90 bpm”, respectively. Therefore, after the processor 120 confirms the relative relationship between the two similar feature data and the target feature data S12 (“the target angle between the forearm and the upper arm is 60 degrees”), the processor 120 estimates the control physiological data (e.g., “heart rate 80 bpm”) according to the two similar feature data and the corresponding two reference physiological signals.

Next, after obtaining the control physiological data, the processor 120 calculates the target fatigue data according to the control physiological data, the correlation parameters and a corresponding part of the reference fatigue data. For example, if the calculated control physiological data is “heart rate 80 bpm”, and the normal the heart rate is 60-100 bpm, the target fatigue data can be “normal”, or the target fatigue data can be directly displayed as “heart rate 80 bpm”.

In some embodiments, the fatigue data generation system 100 periodically performs the above steps S401-S404 to record the corresponding target fatigue data for each time point. For example, when the target human body 200 starts a work at a first time point, the recorded target fatigue data is “a first target fatigue data”. One hour after the target human body 200 performs this work, the recorded target fatigue data is “a second target fatigue data”. In step S405, the processor 120 calculates a relative ratio between the first target fatigue data and the second target fatigue data to obtain a target fatigue index.

For example, If the first target fatigue data is “heart rate 80 bpm” and the second target fatigue data is “heart rate 110 bpm”, the degree of change is 30 bpm, which is equivalent to 37.5% of 80 bpm. Therefore, the target fatigue index can be “fatigue level 37.5%”. In other words, the fatigue data generation system 100 calculates the fatigue level by comparing the initial physiological state of the target human body 200 with the estimated current physiological state.

In some other embodiments, if the target fatigue data is an EMG signal, the processor 120 first performs Fourier transform on the first target fatigue data, and then performs the normal integral to the converted signal, so as to obtain a corresponding first median frequency. Similarly, the processor 120 can transform the second target fatigue data to obtain a second median frequency. Accordingly, the target fatigue index will be obtained with the following formula:

Target fatigue index=(first median frequency−second median frequency)÷first median frequency

In some embodiments, the processor 120 further defines multiple fatigue intervals (e.g., normal, mild, moderate, severe) according to the different proportions of the fatigue level. In other words, the calculated relative ratio (e.g., the value of ratio) corresponds to different fatigue intervals, and fatigue intervals corresponds to different colors. The processor 120 obtains one corresponding color of the different colors according to the calculated target fatigue index, and displays the target fatigue index by the display device D11. For example, the fatigue interval includes “slight fatigue”, “moderate fatigue”, “severe fatigue”. The relative ratios corresponding to these three level are “0-70%”, “70-80%”, “more than 80%”, and each interval further corresponds to a different color/light “green, yellow, red”. If the ratio corresponding to the target fatigue index is “70%”, the display device D11 will display by a yellow light. Accordingly, person under test or system administrator can be clearly informed of the current fatigue state.

The elements, method steps, or technical features in the foregoing embodiments may be combined with each other, and are not limited to the order of the specification description or the order of the drawings in the present disclosure.

It will be apparent to those skilled in the art that various modifications and variations can be made to the structure of the present disclosure without departing from the scope or spirit of the present disclosure. In view of the foregoing, it is intended that the present disclosure cover modifications and variations of this present disclosure provided they fall within the scope of the following claims.

What is claimed is:

1. A fatigue data generation method, comprising:
obtaining, by a camera device, a target image;
obtaining, by a processor, a target feature data from the target image, and inputting the target feature data to a fatigue analysis model which stored in a storage unit, wherein the fatigue analysis model comprises a plurality of reference physiological signals, a plurality of reference feature data, a plurality of reference fatigue data and a plurality of correlation parameters; and
generating a target fatigue data according to the target feature data, the plurality of reference feature data and the plurality of correlation parameters.

2. The fatigue data generation method of claim 1, wherein the plurality of correlation parameters is configured to establish a plurality of first correspondences between the plurality of reference feature data and the reference physiological signals, establish a plurality of second correspondences between the plurality of reference physiological signals and the plurality of reference fatigue data, and generating the target fatigue data comprises:
comparing the target feature data with the plurality of reference feature data to select at least one similar feature data among the plurality of reference feature data;
generating a control physiological data according to the at least one similar feature data, the plurality of correlation parameters and a part of the plurality of reference physiological signals; and
generating the target fatigue data according to the control physiological data, the plurality of correlation parameters and a part of the plurality of reference fatigue data.

3. The fatigue data generation method of claim 2, further comprising:
calculating a relative ratio between a first target fatigue data and a second target fatigue data in the target fatigue data to obtain a target fatigue index, wherein the first target fatigue data corresponds to a first time point, and the second target fatigue data corresponds to a second time point after the first time point.

4. The fatigue data generation method of claim 3, wherein the relative ratio corresponds to one of a plurality of fatigue intervals, and the plurality of fatigue intervals correspond to a plurality of different colors, and the fatigue data generation method further comprises:
obtaining one of the plurality of different colors according to the relative ratio; and
displaying the target fatigue index in the one of the plurality of different colors by a display device.

5. The fatigue data generation method of claim 1, wherein each of the plurality of reference feature data comprises a plurality of reference angles, the plurality of reference angles are calculated according to a plurality of reference parts of a target human body in a reference image.

6. The fatigue data generation method of claim 1, wherein obtaining the target feature data from the target image comprises:
calculating at least one target angle according to a plurality of target parts of a target human body in the target image.

7. The fatigue data generation method of claim 6, wherein calculating the at least one target angle according to the plurality of target parts of the target human body in the target image comprises:
identifying the target image to generate a human body skeleton, wherein the human body skeleton comprises a plurality of part nodes and a plurality of part coordinates corresponding to the target parts; and
generating the at least one target angle according to the plurality of part nodes and the plurality of part coordinates as the target feature data.

8. The fatigue data generation method of claim 7, wherein generating the at least one target angle according to the plurality of part nodes and the plurality of part coordinates comprises:
calculating the plurality of part nodes and the plurality of part coordinates to generate the at least one target angle by law of cosines.

9. The fatigue data generation method of claim 1, further comprising:
obtaining, by the camera device, a reference image to obtain and to establish the plurality of reference feature data; and
obtaining and establishing, by a physiological signal sensor, the plurality of reference physiological signals, wherein the physiological signal sensor is arranged on a target human body; and
establishing a plurality of first correspondences between the plurality of reference feature data and the reference physiological signals according to the plurality of reference physiological signals and the plurality of reference feature data corresponding to a same time.

10. The fatigue data generation method of claim 1, further comprising:
obtaining and establishing, by a physiological signal sensor, the plurality of reference physiological signals, wherein the physiological signal sensor is arranged on a target human body; and
establishing a plurality of second correspondences between the plurality of reference physiological signals and the plurality of reference fatigue data by a conversion formula, wherein one of the plurality of reference physiological signals comprises at least one of an electromyogram, an electrocardiogram, a heart rate, a muscle strength and a blood pressure.

11. A fatigue data generation system, comprising:
a camera device configured to obtain a target image;
a storage unit configured to store a fatigue analysis model, wherein the fatigue analysis model comprises a plurality of reference physiological signals, a plurality of reference feature data, a plurality of reference fatigue data and a plurality of correlation parameters; and
a processor communicatively connected to the camera device and the storage unit, and configured to:
receive the target image to obtain a target feature data from the target image; and
generate a target fatigue data according to the target feature data, the plurality of reference feature data, the plurality of reference physiological signals and the plurality of correlation parameters.

12. The fatigue data generation system of claim 11, wherein the plurality of correlation parameters is configured to establish a plurality of first correspondences between the plurality of reference feature data and the reference physiological signals, establish a plurality of second correspondences between the plurality of reference physiological signals and the plurality of reference fatigue data, and the processor is further configured to:
compare the target feature data with the plurality of reference feature data to select at least one similar feature data among the plurality of reference feature data;
generate a control physiological data according to the at least one similar feature data, the plurality of correlation parameters and a part of the plurality of reference physiological signals; and
generate the target fatigue data according to the control physiological data, the plurality of correlation parameters and a part of the plurality of reference fatigue data.

13. The fatigue data generation system of claim 12, wherein when the processor is configured to generate the target fatigue data according to the control physiological data, the plurality of correlation parameters and the part of the plurality of reference fatigue data, the processor is further configured to:
calculate a relative ratio between a first target fatigue data and a second target fatigue data in the target fatigue data to obtain a target fatigue index, wherein the first target fatigue data corresponds to a first time point, and the second target fatigue data corresponds to a second time point after the first time point.

14. The fatigue data generation system of claim 13, wherein the relative ratio corresponds to one of a plurality of fatigue intervals, and the plurality of fatigue intervals correspond to a plurality of different colors, and the fatigue data generation system further comprises:
a display device coupled to the processor, and configure to display the target fatigue index in one of the plurality of different colors.

15. The fatigue data generation system of claim 11, wherein each of the plurality of reference feature data comprises a plurality of reference angles, the plurality of reference angles are calculated according to a plurality of reference parts of a target human body in a reference image.

16. The fatigue data generation system of claim 11, wherein the processor is further configured to calculate at least one target angle according to a plurality of target parts of a target human body in the target image.

17. The fatigue data generation system of claim 16, wherein the processor is further configured to identify the target image to generate a human body skeleton, wherein the human body skeleton comprises a plurality of part nodes and a plurality of part coordinates corresponding to the target parts; and
wherein the processor is further configured to generate the at least one target angle according to the plurality of part nodes and the plurality of part coordinates as the target feature data.

18. The fatigue data generation system of claim 17, wherein the processor is further configured to calculate the plurality of part nodes and the plurality of part coordinates to generate the at least one target angle by law of cosines.

19. The fatigue data generation system of claim 11, wherein
a physiological signal sensor arranged on a target human body, communicatively connected to the processor and the storage unit to establish the plurality of reference physiological signals;
wherein the camera device is further configured to obtain a reference image to obtain and to establish the plurality of reference feature data; and
wherein the processor is further configured to:
establish a plurality of first correspondences between the plurality of reference feature data and the reference physiological signals according to the plurality of reference physiological signals and the plurality of reference feature data corresponding to a same time.

20. The fatigue data generation system of claim 11, wherein
a physiological signal sensor arranged on a target human body, communicatively connected to the processor and the storage unit to establish the plurality of reference physiological signals; and
wherein the processor is further configured to:
establish a plurality of second correspondences between the plurality of reference physiological signals and the plurality of reference fatigue data by a conversion formula, wherein one of the plurality of reference physiological signals comprises at least one of an electromyogram, an electrocardiogram, a heart rate, a muscle strength and a blood pressure.
